(19) United States
(12) Patent Application Publication
(10) Pub. No.: US 2006/0264996 A1
(43) Pub. Date: Nov. 23, 2006

(54) Lancing device with multi-lancet magazine

(75) Inventors: Richard W. LeVaughn, Newman, GA (US); Gwenn E. Kennedy, Ellenwood, GA (US); Stephen J. Flynn, Peachtree City, GA (US); Carl E. Griffin, Marietta, GA (US); John C. Irwin, Woodstock, GA (US); Mary Kate Pynes, Dallas, GA (US); Stephanie J. Campbell, Kennesaw, GA (US); Christopher J. Ruf, Marietta, GA (US); Mitchell A. Solis, Cumming, GA (US); Avi M. Robbins, Longwood, FL (US); Jason R. Heath, Marietta, GA (US); Ray Lathrop, Atlanta, GA (US); Venkata Katragadda, Kennesaw, GA (US)

Correspondence Address: GARDNER GROFF SANTOS & GREENWALD, P.C., 2018 POWERS FERRY ROAD, SUITE 800, ATLANTA, GA, 30339, US

(73) Assignee: FACET TECHNOLOGIES, LLC, MARIETTA, GA (US)

(21) Appl. No.: 10/569,031

(22) PCT Filed: Aug. 19, 2004
(86) PCT No.: PCT/US04/26982

§ 371 Date: Feb. 17, 2006

Related U.S. Application Data
(60) Provisional application No. 60/497,023, filed on Aug. 20, 2003.

(51) Int. Cl. A61B 17/32

(52) U.S. Cl. 606/181

(57) ABSTRACT

A lancing device and replaceable multi-lancet cartridge for use in sampling of body fluids. Sequential lancets of the cartridge are translationally retracted along a storage path, pivoted into an angularly offset firing path, and translationally propelled by a drive mechanism to lance the subject's skin.

### CROSS-REFERENCE TO RELATED APPLICATION

This application claims the benefit of U.S. Provisional Patent Application Ser. No. 60/497,023, filed Aug. 20, 2003, the entire content of which is hereby incorporated herein by reference for all purposes.

### TECHNICAL FIELD

The present invention relates generally to medical devices and procedures, and more particularly to lancing devices and multi-lancet cartridge assemblies for the collection and/or analysis of samples of blood or other bodily fluids.

### BACKGROUND OF THE INVENTION

Many medical procedures require puncturing of the skin, and sometimes underlying tissues, of an animal or human subject. For example, a sharp lancet tip is commonly used to puncture the subject's skin at a lancing site to obtain a sample of blood, interstitial fluid or other body fluid, as for example in blood glucose monitoring by diabetics, and in blood typing and screening applications. In some instances, a person must periodically sample their blood for multiple testing throughout the day or week. Because re-use of a lancet can result in infection or spread of bloodborne contaminants, persons requiring repeated testing often must carry multiple lancets with them. This can be inconvenient and lead to reduced compliance with a prescribed test regimen. Accordingly, it can be seen that needs exist for a convenient, compact multi-use lancing device.

Various devices are known for sampling blood and other body fluids for analysis of the condition of a human or other animal subject. For example, U.S. Pat. No. 5,971,941 is understood to show a cassette with test strips for placement by a slider. A lancet pierces the skin surface so that blood can be obtained for analysis. The lancets are integrated on a test strip, and are positioned together with the test strip. Another embodiment is understood to show a disposable cylindrical insert having a lancet and a test membrane with an aperture for the lancet. The insert is inserted in a mounting cavity of a plunger or piston, which forces the lancet outward for blood withdrawal. DE 198 19 407 A1 is understood to show a multiplicity of test strips with integrated lancets for insertion into an analysis device.

U.S. Pat. No. 4,787,398 is understood to show a device with a plunger for directing a lancet outward, and has an evaluation system and a display system. A replaceable unit is applied to the device for each measurement. The replaceable unit comprises the lancet and a test strip, which is wetted with blood. This replaceable unit is thrown away after each use. EP 0 449 525 A1 is understood to show a blood withdrawal system wherein a new lancet is inserted manually into a release device before each use. A test strip is then inserted into the device. U.S. Pat. No. 4,627,445 is understood to show a device for measuring blood sugar, with an integrated blood withdrawal unit. A new replaceable lancet and test elements must be installed to the device for testing, and afterward disassembled. U.S. Pat. No. 5,951,492 is understood to show a disposable unit with a capillary tube and a test strip, to which sampled blood taken is applied. The capillary tube includes a lancet. A new disposable unit is attached and removed before and after each measurement.

EP 0877250 A2, EP 0949506 A2 and EP 811843 A2 are understood to show devices having a multiplicity of test elements arranged on a rotatable disk carrier. The test elements are brought successively into a working position and pushed out of the housing to be wetted with blood. U.S. Pat. No. 6,228,100 and U.S. Pat. No. 4,794,926 are understood to show lancets arranged on a carrier, which is rotated with respect to a housing.

German Application DE 100 57 832 C1 is understood to show a lancing device of a known form. Other lancing devices understood to include multiple lancets are shown, for example, in U.S. Patent App. Serial No. 2002/0087056 A1 and WO 02/36010 A1. EP 0589186 B1 is understood to show a lancet with a removable protective cap. WO 01/66010 A1 is understood to include a multiplicity of lancets in a magazine, with an opening of the chamber closed by an elastic material, which is penetrated in the puncture process.

Known sampling devices have, however, not proven fully satisfactory to all users for a variety of reasons. Accordingly, it has been discovered that needs exist for an improved lancing device capable of carrying out multiple sampling procedures without the need for separately loading individual lancets. It has also been discovered that needs exist for a convenient, disposable multi-lancet cartridge that can be loaded into a multi-use lancing device for carrying out multiple sampling procedures and be removed and replaced when fully or partially spent or when replacement is otherwise desired. It is to the provision of an improved lancing and/or sampling device, and a lancet cartridge for such a device, meeting these and other needs that the present invention is primarily directed.

### SUMMARY OF THE INVENTION

Briefly described, in one aspect, the present invention is a lancing device having an outer housing for receiving a replaceable multi-lancet cartridge or magazine. In one embodiment, the lancet cartridge or magazine remains stationary relative to the housing and drive mechanism of the lancing device, and the drive mechanism is advanced sequentially along the cartridge or magazine within the outer housing for carrying out multiple lancing procedures.

In another embodiment, the drive mechanism is stationary and the cartridge or magazine advances sequential lancets through the drive mechanism. The cartridge or magazine preferably includes a top and bottom for retaining the array of lancets sequentially mounted within the cartridge and for constraining the active lancet along a controlled and pre-defined path of travel during the lancing stroke. The cartridge or magazine preferably also includes a mechanism for retaining protective endcaps that have been removed from the lancets in a position out of the path of travel of the lancets and for preventing the caps from rattling within the housing.

In another aspect, the invention is a lancing device having a housing for receiving a plurality of lancets, and a drive mechanism translationally mounted within the housing for traversing the plurality of lancets and sequentially engaging individual lancets from the plurality of lancets, and driving the engaged lancet along a path of travel.

In still another aspect, the present invention is a lancing device including a drive mechanism for sequentially engaging individual lancets from a multi-lancet cartridge, retracting the engaged lancet along a first path of travel, and advancing the lancet along a second path of travel, wherein the drive mechanism preferably includes a coupling allowing the engaged lancet to pivot between the first and second paths of travel.

In another aspect, the invention is a lancing device including a plurality of lancets, wherein the plurality of lancets are initially joined together in an attached array, and wherein the lancing device preferably further includes a separating member for sequentially detaching individual lancets from the attached array.

In yet another aspect, the invention is a magazine for a lancing device, the magazine preferably including a plurality of lancets, and guide surfaces defining a storage position and a firing position for each of the plurality of lancets.

In another aspect, the invention is a magazine for a lancing device, the magazine including a lancet having a sharp tip portion, and being movable between a retracted position and a lancing position. The magazine preferably further includes a window through which the sharp tip portion of the lancet projects in its lancing position, and a shutter movable between an open position allowing passage of the sharp tip portion of the lancet through the window and a closed position blocking the window.

In another aspect, the invention is a lancet cartridge including a plurality of lancets, each lancet having a body and a sharp tip projecting from the body. The lancet cartridge preferably further includes a common sterility block into which the sharp tips of each of the plurality of lancets is removably embedded.

In still another aspect, the invention is a lancet cartridge including a plurality of lancets, each of the plurality of lancets being joined to at least one adjacent lancet by a separable segment.

In another aspect, the invention is a lancet including a body having first and second ends, and a sharp tip projecting axially from a first end of the lancet body, wherein the second end of the lancet body is flared and comprises an arcuate distal surface.

Example embodiments of the present invention include an improved sampling device that is convenient, compact, and includes multiple lancets in a single cassette or cartridge. The present invention preferably increases convenience for the user, thereby encouraging more frequent testing and insuring compliance with the subject's prescribed testing regimen.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a lancing device according to an example embodiment of the present invention, with the drive mechanism in a first position.

FIG. 2 is a perspective view of the device of FIG. 1, after advancement of the drive mechanism to a subsequent position, with a top cover portion removed to show the lancet magazine and internal workings including the drive mechanism.

FIGS. 3a-3d are perspective, side, top and end views of a lancet cartridge according to an example embodiment of the present invention.

FIGS. 4a-4c are perspective, end, and partial cutaway top views of a lancet magazine according to an example embodiment of the present invention

FIGS. 5-7 show a sequence of operation of a lancing device and lancet magazine according to an example embodiment of the present invention.

FIG. 8 shows a lancing device according to another example embodiment of the present invention.

FIG. 9 shows another embodiment of a lancet magazine or cartridge according to the present invention.

FIG. 10 is a detailed view of a single lancet of the lancet magazine of FIG. 9.

FIGS. 11a-11c show a sequence of operation of a lancing device and lancet according to FIG. 10.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

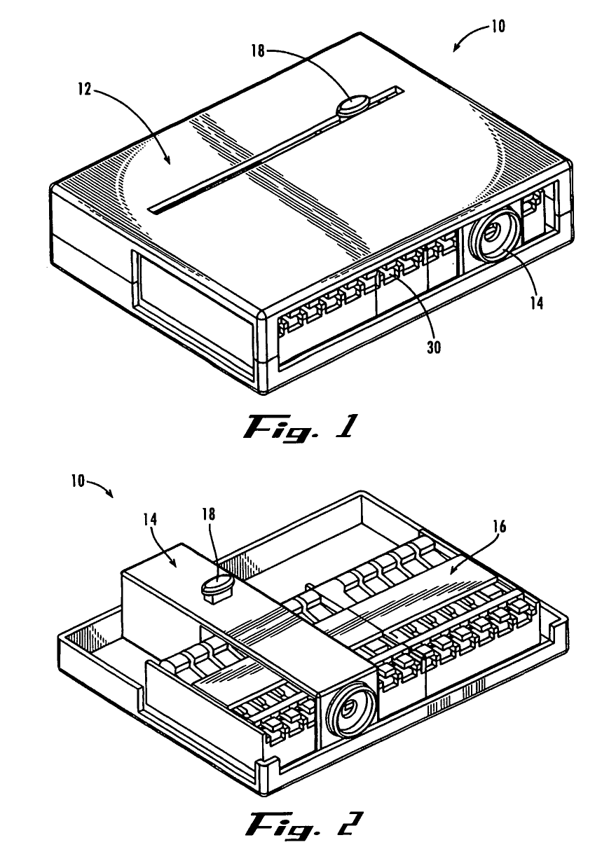

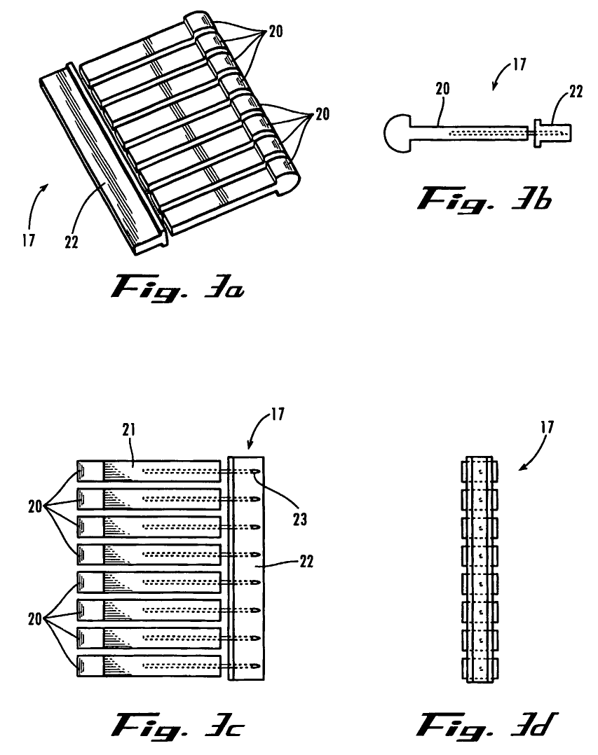

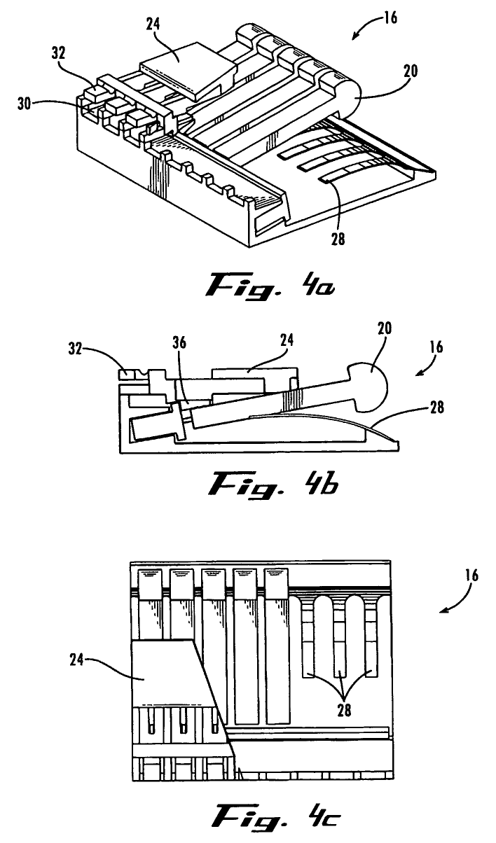

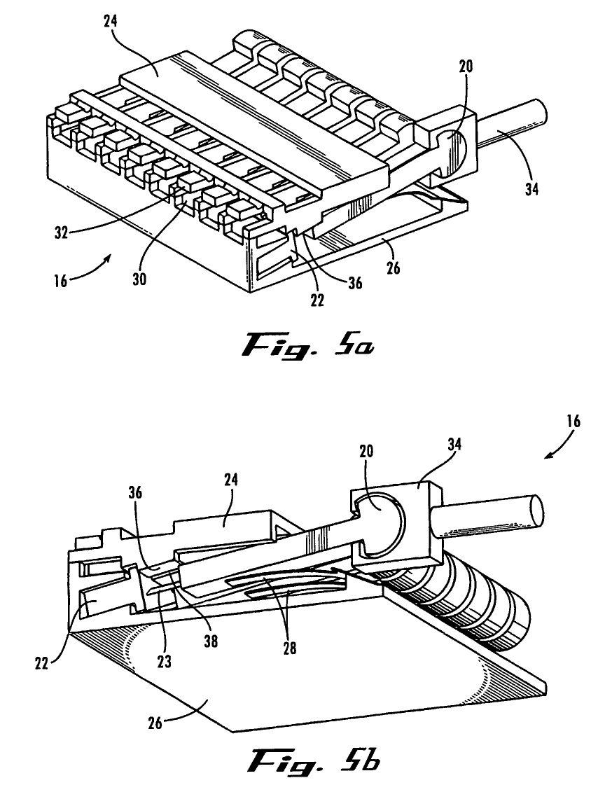

With reference now to the drawing figures, which form a part of this disclosure and are hereby incorporated by reference as a part of this detailed description, example embodiments of the present invention are shown and described. FIGS. 1 and 2 show a lancing device 10 according to an example embodiment of the present invention. The lancing device 10 preferably generally comprises a housing 12 and a drive mechanism 14. A multi-lancet magazine 16, for example as shown in FIG. 4, is preferably removably loaded into the lancing device 10 for sequential engagement of the lancets with the drive mechanism. The housing 12 and drive mechanism 14 are preferably re-usable, and the lancet magazine 16 is preferably a disposable component, so that spent magazines can be removed for disposal and replaced with new lancet magazines. In alternate embodiments, a lancet cartridge portion 17 of the magazine 16, as shown in FIG. 3, is removable from the magazine, and the magazine is reusable with replacement lancet cartridges.

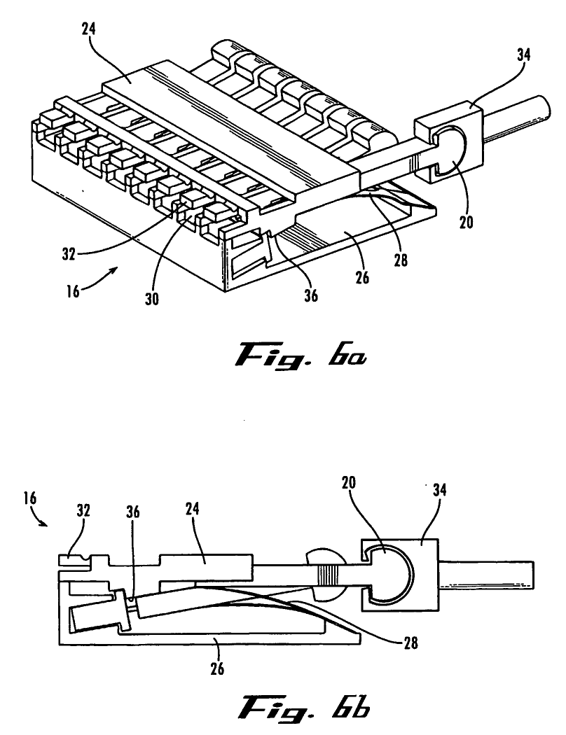

The drive mechanism 14 preferably includes a piston with a coupling 34 for sequentially engaging individual lancets of the magazine or cartridge. As seen best in FIGS. 5-7, the coupling of the piston 34 preferably comprises a C-shaped channel or other configuration having a narrow mouth portion and a wider arcuate distal portion, so that the coupling can slide translationally into and out of engagement with sequential lancets of the magazine or cartridge, and securely engage flared end portions of the lancets for axial advancement and return of the lancets through their path of travel along a lancing stroke. Preferably, the engagement of the coupling 34 with the flared end of the lancet permits a degree of pivotal motion of the lancet within the coupling, thereby allowing the lancet to rotate between angularly-offset first and second travel paths as described below. The drive mechanism 14 preferably further comprises an activation mechanism coupled to the piston 34 for driving the piston and an active lancet engaged within the coupling of the piston through the path of travel of the lancing stroke, from a first “retracted” position shielded within the housing, through a second “lancing” position wherein a tip portion of the active lancet projects outwardly of the housing for lancing the subject's skin at a sampling site, and back to a third “return” position shielded within the housing. The activation mechanism can drive the piston and active lancet via spring-driven or other biasing means, a ratchet system, manual advancement mechanism, a screw type drive, or other means of propulsion. The drive mechanism 14 preferably also comprises an actuator or trigger 18 for allowing the user to selectively actuate the activation mechanism to drive the active lancet through the lancing stroke.

The housing 12 is preferably a generally rigid shell serving to house and protect the drive mechanism 14 and the magazine 16.. The housing 12 can be integrally formed or can comprise multiple pieces that are adjoined together. The housing is preferably fabricated from plastic, metal or other substantially rigid and durable material of construction. In preferred form, the housing comprises a base portion to which the drive mechanism is mounted, and a top cover portion that is permanently or releasably secured to the base portion. In an example embodiment, the housing is a generally rectangular box having a top panel, a bottom panel, a front panel, a back panel, and first and second side panels. The front panel preferably comprises a least one lancet opening through which the tip of the active lancet projects in its lancing position. Optionally, a plurality of lancet openings or an elongate slotted lancet opening is provided. The top panel preferably comprises an opening through which the actuator 18 projects. One or both side panels preferably comprise access ports for inserting and/or removing lancet magazines or cartridges into and out of the lancing device.

In one example embodiment, the drive mechanism 14 traverses a path from one side of the housing to the other, for sequentially engaging individual lancets of a lancet magazine 16, which is fixed in position relative to the housing 12. For example, FIG. 1 shows the drive mechanism 14 in a first position toward a first side of the housing, for engagement with a lancet toward the first side of the magazine 16; and FIG. 2 shows the drive mechanism in a second position toward the second side of the housing, for engagement with another lancet toward the second side of the magazine. The drive mechanism 14 can be manually or automatically advanced along its path of traverse. A forward portion of the drive mechanism 14 preferably extends through the lancet opening in the front panel of the housing, and defines a contact ring surrounding a central opening for contact against the skin at the lancing site. In the depicted embodiment, the forward portion of the drive mechanism traverses the lancet opening in the front panel of the housing as the remainder of the drive mechanism traverses the housing to engage sequential lancets. In an alternate embodiment, the drive mechanism 14 remains stationary within the housing, and the lancet magazine 16 is manually or automatically advanced through the drive mechanism 14 to engage sequential lancets of the cartridge with the drive mechanism. For example, a linear array of lancets in a belt-like cartridge can be fed in through the access port in one side of the housing and discharged out of the access port in the other side, as the cartridge is advanced through the stationary drive mechanism.

FIGS. 3a- 3d show a linear array of lancets 20 in a belt-like cartridge. The lancets of a cartridge are preferably oriented with their sharp tips aligned generally in the same direction, in a planar linear array. Alternatively, the lancets of a cartridge are arranged in a conical array, as described below, or in a curved or otherwise configured array. Preferably, the lancet body 21 is formed of plastic and is injection-molded around a piercing element 23 having a sharp needle or blade tip projecting from its proximal end. Each lancet preferably comprises a flared distal end, having a D-shaped profile with an arcuate distal face, for engagement with the coupling of the drive mechanism, and a sharp tip projecting from its proximal end. One or more protective endcap members 22 are preferably provided for covering the sharp tips of the lancets to maintain sterility and prevent inadvertent needle sticks during loading and unloading. In the depicted embodiment, a single unitary or common sterility block 22 forms the endcap member for all of the lancets 20 of the cartridge. In alternate embodiments, each lancet has a separate, individual sterility cap. The capacity of a linear magazine or cartridge can optionally be doubled by attaching two such magazines together end-to-end, either mechanically or in design, into an adjoined magazine. For example, after all lancets on a first end portion of the adjoined magazine are used, the magazine is ejected, turned over and lancets on a second end portion are used.

FIGS. 5-7 show a sequence of operation of a multi-lancet magazine assembly 16 and a lancing device according to the present invention. The magazine preferably comprises a cartridge having a linear array of lancets 20 therein, the lancets initially being positioned in an inclined storage position with their sharp tips embedded in a sterility cap 22. The magazine preferably further comprises a magazine top 24 and a magazine bottom 26, defining first and second travel paths for each lancet 20. The cartridge preferably also includes one or more cantilevered leaf springs or other biasing element(s) 28, for moving an active lancet from its initially inclined storage position (see FIG. 4) into a firing position (see FIGS. 6 and 7). The storage position is preferably oriented at an acute angle relative to the firing position, most preferably at an angle of between about 5° to about 30° beneath the firing position.

FIG. 5 shows engagement of an active lancet 20 with the coupling of the drive mechanism piston 34, to retract the lancet from the sterility block 22 along the first travel path. The biasing element 28 pushes the top of the lancet body 21 against a shelf 36 on the underside of the magazine top 24. The shelf 36 preferably comprises an angled surface extending rearwardly from the sterility block 22, a distance approximately equal to or slightly greater than the length of the lancet tip 23 extending from the lancet body 21, constraining movement of the lancet to linear translation along the first travel path as the lancet is axially (i.e., in the direction of the lengthwise axis of the lancet needle) withdrawn from the sterility block. In this manner, the shelf 36 prevents bending damage to the lancet tip 23 by holding the active lancet 20 in its downward (storage/pre-fire) position until the lancet tip is completely withdrawn from the sterility block 22, at which point the edge of the lancet body 21 clears the end of the shelf 36, allowing the biasing element 28 to rotate the active lancet 20 into its generally horizontal firing position, shown in FIG. 6. The lancet tip 23 preferably passes through a slot 38 formed in the shelf 36 as the lancet moves from the storage position to the firing position.

The active lancet 20 is driven along a second travel path, between a retracted position shown in FIG. 6 and a lancing position shown in FIG. 7, the second travel path preferably being offset at an acute angle, and more preferably at an angle of between about 5° to about 30°, above the first travel path. The lancet preferably then returns along the second travel path, from its lancing position to its return position. The second travel path is preferably generally horizontal (i.e., generally parallel to the magazine bottom and the bottom panel of the housing, and is defined by a channel or pathway along a bottom surface of the magazine top 24. When the active lancet 20 is in its lancing position, its sharp tip projects through a window or opening 30 in a front surface of the magazine 16. Optionally, the magazine comprises one or more stops adjacent the opening 30 for contacting a cooperating surface feature of the lancet to limit its travel and thereby control lancing penetration depth. A door or shutter 32 preferably extends from the magazine adjacent the opening 30, and is hingedly movable from an initial open position allowing passage of the lancet tip to a closed position blocking the opening 30 to prevent inadvertent contact with a used lancet. Optionally, the door 32 includes one-way locking features such as a barb for engagement with a cooperating detent formed in the magazine adjacent the opening 30 to prevent re-opening the door after closure, and thereby prevent re-use of a lancet.

In operation, the user preferably opens an access port in the housing of the lancing device, loads a preassembled multi-lancet cartridge into the lancing device, and closes the housing. A first unused lancet is engaged with the drive mechanism to become the active lancet. The drive mechanism is cocked or otherwise energized, if necessary, and the lancet is withdrawn along its first travel path to retract its tip from the protective endcap or sterility block. The biasing element applies force to rotate the lancet from its storage position to its firing position. The lancing device is positioned against a finger or other lancing site on the subject's body, and the drive mechanism is actuated to propel the lancet along its second travel path to lance the skin at the lancing site. The lancet then retracts along the second travel path to its returned position. The lancing device is advanced to release the used lancet and bring another lancet into the active position in engagement with the drive mechanism. After all lancets of a cartridge have been used, the spent cartridge is removed from the lancing device, and a fresh cartridge is installed.

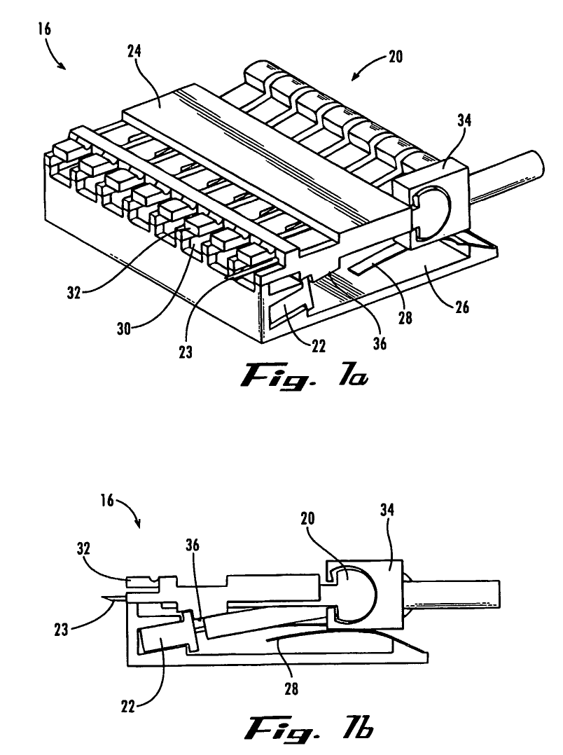

FIG. 8 shows a blood sampling device 110 according to another example embodiment of the invention. The device includes a reusable housing 1 12, an actuator and drive mechanism 114, and a removable and replaceable lancet cartridge 116. The cartridge preferably comprises a linear array of attached lancets 18 for sequential firing. Adjacent individual lancets of the cartridge are preferably attached to one another by intermediate webs, or are integrally formed and directly attached to one another by separable neck segments. An active lancet of the cartridge is preferably advanced into a firing position by an advancing arm 124, into engagement with the drive mechanism. A blade or other inclined separating member 120 is driven between the active lancet and the adjacent lancet to separate the active lancet from the cartridge. Preferably, the actuation of the separating member also serves to simultaneously or sequentially energize the drive mechanism, as by compressing a drive spring and engaging a trigger mechanism. The trigger mechanism is released, whereupon the drive mechanism propels the active lancet through its travel path, between a retracted position within the housing to a lancing position wherein at least a tip portion of the lancet extends out of the housing to lance the subject's skin at a lancing site, and preferably back to a returned position within the housing. The advancing mechanism can then be actuated to eject the used lancet from an ejection port 122 of the housing, and bring the next lancet of the cartridge into the active position for subsequent use.

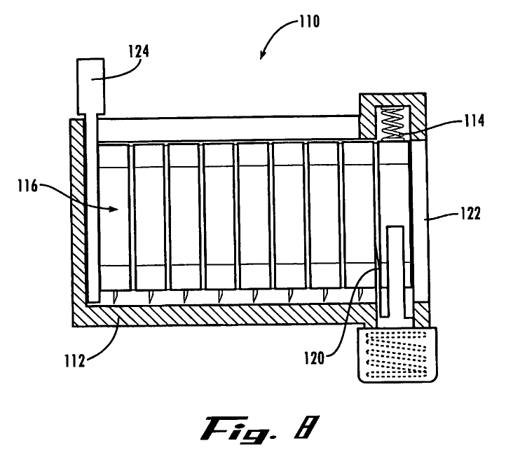

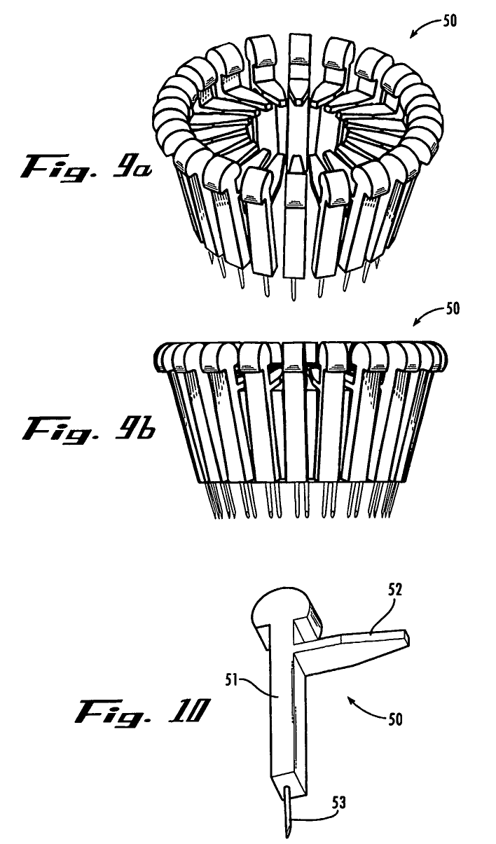

Although the invention has been described primarily with reference to lancet cartridges comprising a linear array of lancets, alternate embodiments of the invention include conical, cylindrical or otherwise configured curved arrays of lancets. FIG. 9 shows a generally conical arrangement of lancets 50 for a lancet cartridge. With reference now to FIG. 10, each individual lancet 50 preferably comprises a lancet body 51, a sharp tip 53 such as a needle or blade extending axially from a first end of the lancet body, a biasing arm 52 extending transversely from a side of the lancet body generally perpendicular to the lengthwise axis of the lancet body, and a flared second end preferably having a distal face with an arcuate profile. The lancets are preferably assembled in a circular array within a housing, with their axes inclined inwardly so that their tips are directed toward the central axis of the circular array, to form a conical magazine or cartridge.

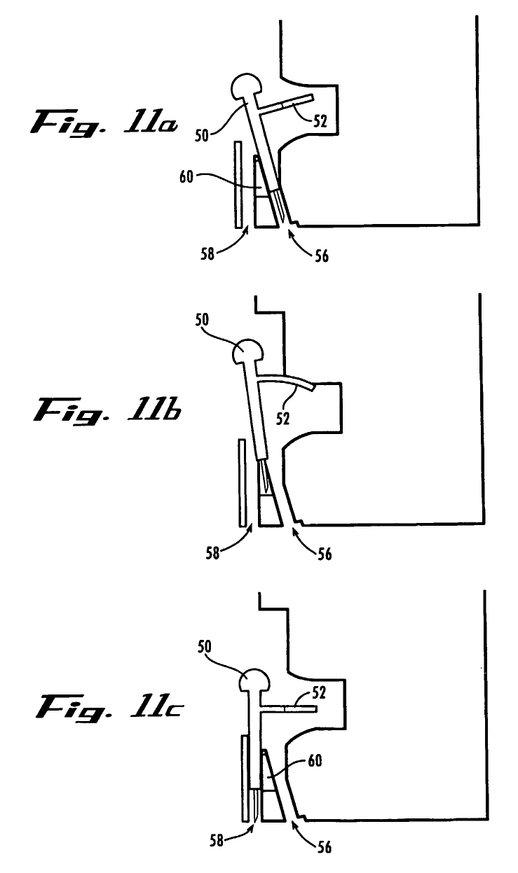

FIGS. 11a, 11b and 11c show a sequence of operation of a lancet 50 of the type shown in FIG. 10. The lancet 50 is initially disposed in a storage position, with its lengthwise axis aligned in a first angular orientation, preferably within a storage channel 56 or other guide surface(s) of the cartridge or lancing device. The lancet 50 is engaged by the drive mechanism of the lancing device, and is translationally retracted along a first travel path defined by the storage channel 56, as shown in FIG. 11 a, into a retracted position. At the retracted position, the biasing arm 52 engages a biasing surface of the cartridge or lancing device, as shown in FIG. 11b, causing the lancet to rotate into a second angular orientation, preferably within a firing channel 58 of the cartridge or lancing device. A shelf positioned between the storage channel 56 and the firing channel 58 preferably constrains the lancet to translational motion along its first travel path, until the lancet body is retracted clear of the shelf. The shelf preferably defines a slot 60, through which the lancet tip 53 passes as the lancet rotates into its second angular orientation under the influence of the biasing arm 52. The drive mechanism is then actuated to translationally propel the lancet 50 in its axial direction, along a second travel path defined by the firing channel 58, from the retracted position to a lancing position wherein the lancet tip projects from the lancing device to lance the subject's skin at a lancing site, and preferably back again to a return position within the lancing device. The first and second travel paths are preferably offset from one another at an acute angle, and more preferably at an angle of between about 5° to about 30°.

While the invention has been described with reference to preferred and example embodiments, it will be understood by those skilled in the art that a variety of modifications, additions and deletions are within the scope of the invention, as defined by the following claims.

What is claimed is:

1. A lancing device comprising a housing for receiving a plurality of lancets, and a drive mechanism translationally mounted within said housing for traversing the plurality of lancets and sequentially engaging individual lancets from the plurality of lancets, and driving the engaged lancet along a path of travel.

2. The lancing device of claim 1, wherein the drive mechanism retracts the engaged lancet along a first path of travel and advances the engaged lancet along a second path of travel.

3. The lancing device of claim 2, wherein the first and second paths of travel are angularly offset from one another.

4. The lancing device of claim 3, wherein the first and second paths of travel are offset from one another at an acute angle.

5. The lancing device of claim 4, wherein the first and second paths of travel are offset from one another by an angle of between about 5° to about 30°.

6. The lancing device of claim 2, wherein the plurality of lancets are contained within a magazine, said magazine defining the first and second travel paths.

7. The lancing device of claim 6, wherein said magazine comprises a shelf between the first and second travel paths, said shelf comprising at least one slot for passage of a tip portion of the engaged lancet as the engaged lancet moves between the first and second travel paths.

8. The lancing device of claim 6, wherein said magazine comprises at least one spring for biasing the engaged lancet from the first travel path into the second travel path.

9. The lancing device of claim 6, wherein a common sterility block encapsulates tip portions of the plurality of lancets within said magazine.

10. The lancing device of claim 6, wherein said magazine comprises at least one window through which a tip portion of the engaged lancet projects in a lancing position of the second travel path.

11. The lancing device of claim 10, wherein said magazine comprises at least one shutter, each shutter associated with one of the at least one windows and movable between an open position allowing passage of the tip portion of the engaged lancet through the associated window and a closed position blocking the associated window.

12. The lancing device of claim 2, wherein each of the plurality of lancets comprises a flared end having an arcuate distal face, and wherein the drive mechanism comprises a coupling for engaging the flared end of the engaged lancet and permitting pivoting of the engaged lancet within the coupling.

13. The lancing device of claim 2, wherein each of the plurality of lancets comprises a biasing arm projecting transversely from a body portion of the lancet, for biasing the engaged lancet from the first path of travel to the second path of travel.

14. The lancing device of claim 1, wherein the plurality of lancets are initially joined together in an attached array, and wherein the lancing device further comprises a separating member for sequentially detaching individual lancets from the attached array.

15. A lancing device comprising a drive mechanism for sequentially engaging individual lancets from a multi-lancet cartridge, retracting the engaged lancet along a first path of travel, and advancing the lancet along a second path of travel, wherein the drive mechanism comprises a coupling allowing the engaged lancet to pivot between the first and second paths of travel.

16. The lancing device of claim 15, wherein each lancet of the multi-lancet cartridge comprises a flared end having an arcuate distal face, and wherein the coupling of the drive mechanism comprises a channel having a narrow mouth portion and a wider arcuate distal portion for pivotal engagement of the arcuate distal face of the engaged lancet.

17. The lancing device of claim 15, wherein the multi-lancet cartridge comprises a linear array of lancets.

18. The lancing device of claim 15, wherein the multi-lancet cartridge comprises a conical array of lancets.

19. The lancing device of claim 15, wherein the individual lancets of the multi-lancet cartridge are in a magazine, said magazine comprising guide surfaces defining the first and second paths of travel.

20. The lancing device of claim 19, wherein the magazine comprises a slotted shelf between the first and second paths of travel.

21. The lancing device of claim 19, wherein the magazine comprises at least one biasing element for moving the engaged lancet from the first path of travel to the second path of travel.

22. The lancing device of claim 15, wherein each lancet of the multi-lancet cartridge comprises a body having a sharp tip projecting from a first end thereof, and a biasing arm extending transversely from the body.

23. A lancing device comprising a plurality of lancets, wherein said plurality of lancets are initially joined together in an attached array, and wherein the lancing device further comprises a separating member for sequentially detaching individual lancets from the attached array.

24. The lancing device of claim 23, wherein the separating member comprises a blade.

25. The lancing device of claim 23, wherein actuation of the separating member to detach a lancet from the attached array also operates to energize a drive mechanism of the lancing device.

26. A magazine for a lancing device, said magazine comprising:
a plurality of lancets; and
guide surfaces defining a storage position and a firing position for each of the plurality of lancets.

27. The magazine of claim 26, wherein the storage position and the firing position are offset from one another at an acute angle.

28. The magazine of claim 27, wherein the storage position and the firing position are offset from one another at an angle of between about 5° to about 30°.

29. The magazine of claim 26, wherein the plurality of lancets are arranged in a linear array.

30. The magazine of claim 26, wherein the plurality of lancets are arranged in a conical array.

31. The magazine of claim 26, further comprising at least one biasing member for moving each of the plurality of lancets from the storage position to the firing position.

32. The magazine of claim 31, wherein the biasing members comprise a cantilevered spring arm biased against each of the plurality of lancets.

33. The magazine of claim 31, wherein the biasing members comprise a biasing arm extending transversely from each of the plurality of lancets.

34. A magazine for a lancing device, said magazine comprising:
a lancet having a sharp tip portion, and movable between a retracted position and a lancing position;
a window through which the sharp tip portion of the lancet projects in its lancing position; and
a shutter, pivotal about a generally horizontal axis, and movable between an open position allowing passage of the sharp tip portion of the lancet through the window and a closed position blocking the window.

35. A lancet cartridge comprising a plurality of lancets, each lancet having a body and a sharp tip projecting from the body, the lancet cartridge further comprising a common sterility block into which the sharp tips of each of the plurality of lancets is removably embedded.

36. A lancet cartridge comprising a plurality of lancets, each of the plurality of lancets being joined to at least one adjacent lancet by a separable segment.

37. A lancet comprising a body having first and second ends, and a sharp tip projecting axially from a first end of the lancet body, wherein the second end of the lancet body is flared and comprises an arcuate distal surface.

38. The lancet of claim 35, further comprising a biasing arm extending transversely from a side of the body.
